(19) United States
(12) Patent Application Publication
(10) Pub. No.: US 2021/0147790 A1
(43) Pub. Date: May 20, 2021

(54) METHOD FOR PICKING BIOLOGICAL SAMPLE

(71) Applicant: General Automation Lab Technologies Inc., San Carlos, CA (US)

(72) Inventors: Alexander Hallock, San Carlos, CA (US); Marc Glazer, San Carlos, CA (US)

(21) Appl. No.: 17/091,189

(22) Filed: Nov. 6, 2020

Related U.S. Application Data
(60) Provisional application No. 62/931,338, filed on Nov. 6, 2019.

(51) Int. Cl. C12M 1/00; C12N 1/02; C12M 1/26

(52) U.S. Cl. CPC C12M 47/04; C12M 33/00; C12N 1/02

(57) ABSTRACT

A method of transferring material using a picking pin having a distal tip includes: forming a first coating of organic molecules on at least a portion of the distal tip of the picking pin; dipping at least a portion of the coated distal tip in a first source such that an amount of material is picked up from the first source and attached to the first coating; dipping the coated distal tip attached with the amount of material into a first target so as to release some of the amount of the material to the first target; and removing the first coating from the distal tip. The procedure can be repeated for transferring additional material from the same or different source to the same or different targets.

### CROSS REFERENCE TO RELATED APPLICATION

This application claims priority to U.S. Provisional Application No. 62/931,338, filed Nov. 6, 2019, the disclosure of which is incorporated by reference herein in its entirety.

### BACKGROUND

Microbial cells, such as bacteria, fungi, archaea, or viruses are often used as carriers in genomic research to replicate or express specific genes or proteins. Isolates of particular microbial species or variants are often isolated from mixed microbial populations. When grown in petri dishes, microbial cells usually form colonies. The microbial colonies can be hand-picked using a picker, such as a toothpick, and placed in individual wells of a microwell plate for subsequent incubation, processing or analysis. Automated systems for colony picking also became available in recent years. For example, a picking system being developed by the instant applicant uses a picking pin to pick materials, such as bacterial cells, from high density microwells of microfabricated chips which are used in culturing and screening microbes.

For considerations such as cost and precise calibration, the picker used in highly automated picking systems is typically used repeatedly in picking material from one or more sources. The picking pin (the part that actually physically contacts the source material) can have very small diameter (e.g., <500 microns, or even <100 microns) and in the picking operations may be subject to high impact and repeatedly bent. Further, between successive picking rounds, to avoid cross contamination, the picking pin needs to be cleaned and sterilized to remove any remnants of the previously picked material. The repeated use requirement, coupled with requirements for efficient cleaning and sterilization call for picking pin material to be highly chemically inert, anti-corrosion resistant, high impact and heat resistance in repeated picking and heat-cooling cycles. Metals or metal alloys meeting such stringent requirements can include W and W containing alloys.

Although a metallic picking pin can have a hydrophilic surface, the transfer rate, i.e., the chance or confidence that the material of interest (e.g., cells) picked up by the picking pin can be limited. Sometimes, the picking pin can fail to pick any material of interest (although it may have picked up an amount of liquid from the source). This can lead to problems in the downstream analysis, and may require additional repetitive operations to improve the confidence that material of interest is transferred to the target location. There is a need to improve picking efficiency or transfer rate of the picking pin.

### SUMMARY OF INVENTION

In one aspect, the present disclosure provides a method of transferring material using a picking pin having a distal tip, comprising, in the following order: (a1) forming a first coating of organic molecules on at least a portion of the distal tip of the picking pin; (a2) dipping at least a portion of the coated distal tip in a first source such that an amount of material is picked up from the first source and attached to the first coating; (a3) dipping the coated distal tip attached with the amount of material into a first target so as to release some of the amount of the material to the first target; and (a4) removing the first coating from the distal tip. The method may further include: (b1) forming a second coating of organic molecules on at least a portion of the distal tip of the picking pin (the second coating may have a same or different composition than the first coating); (b2) dipping at least a portion of the distal tip in a second source (the second source may be the same as or different from the first source) such that an amount of material is picked up from the second source and attached to the second coating; (b3) dipping the distal tip attached with the amount of material into a second target (the second target being the same as or different from the first target) so as to release some of the amount of the material to the second target; and (b4) removing the second coating from the distal tip. The method can include further similar cycles of picking and transferring more materials from a source to a target, reusing the picking pin in each cycle, wherein in each cycle the picking pin is coated with a fresh coating and then the coating is stripped away to prepare for the next cycle.

In some embodiments, the first or second coating comprises a polymer. The first or second coating can be hydrophilic or hydrophobic. In some embodiments, the first or second coating comprises PDMS. In some embodiments, the first or second coating comprises PVA.

In some embodiments, forming the first or second coating comprises dipping the distal tip in a polymer solution. In some embodiments, forming the first or second coating comprises dipping the distal tip in two separate solutions: (1) a polymer solution; and (2) a solution containing a crosslinker suitable for crosslinking the polymer in the polymer solution.

Removing the first or second coating from the distal tip can comprise applying heat on the coated distal tip, or dipping the coated distal tip in a chemical solution suitable for dissolving the coating.

In some embodiments, the first or second source comprises at least one cell.

In some embodiments, the first or second source comprises a liquid.

In some embodiments, the first source is contained in a first microcell of a microfabricated chip, and the second source is contained in a second microcell of the microfabricated chip.

In some embodiments, the first source and/or the second source are covered by a cover film, and dipping at least a portion of the coated distal tip into the first and/or second source comprises puncturing through the cover film.

In some embodiments, the first or second target comprises a liquid.

In some embodiments, the first or second target is contained in a well of a microfabricated device or a microplate.

### BRIEF DESCRIPTION OF THE DRAWINGS

FIG. 1 is a flow chart showing steps of an example method of transferring material using a picking pin according to embodiments of the present invention.

FIG. 2 is a schematic depiction of a picking pin according to embodiments of the present invention.

### DETAILED DESCRIPTION

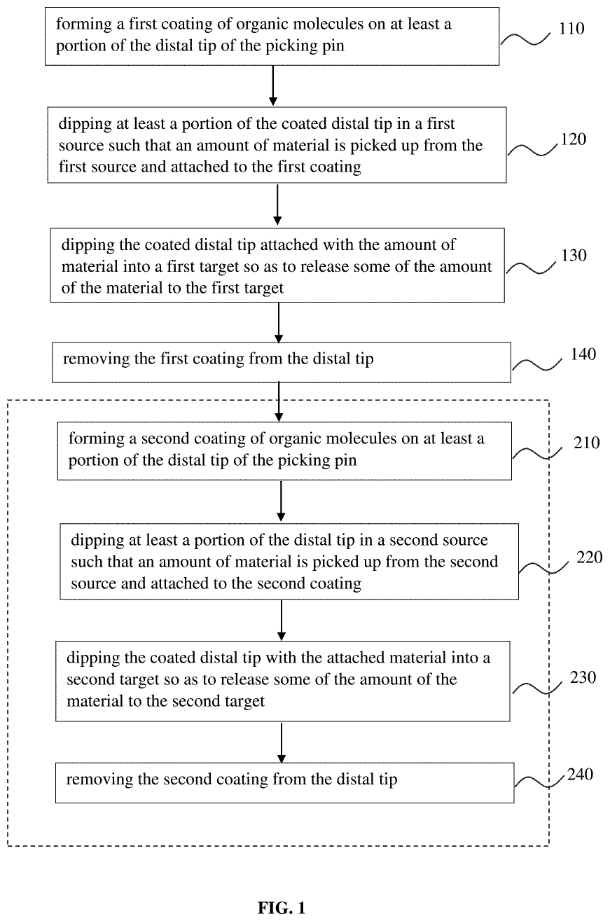

In one aspect, and with reference to FIG. 1, the present disclosure provides a method of transferring material using a picking pin having a distal tip, comprising, in the following order: (a1) at 110, forming a first coating of organic molecules on at least a portion of the distal tip of the picking pin; (a2) at 120, dipping at least a portion of the coated distal tip in a first source such that an amount of material is picked up from the first source and attached to the first coating; (a3) at 130, dipping the coated distal tip attached with the amount of material into a first target so as to release some of the amount of the material to the first target; and (a4) at 140, removing the first coating from the distal tip.

Also referring to FIG. 1, the method may further include, in such order: (b1) at 210, forming a second coating of organic molecules on at least a portion of the distal tip of the picking pin (the second coating may have a same or different composition than the first coating); (b2) at 220, dipping at least a portion of the distal tip in a second source (the second source may be the same as or different from the first source) such that an amount of material is picked up from the second source and attached to the second coating; (b3) at 230, dipping the distal tip attached with the amount of material into a second target (the second target being the same as or different from the first target) so as to release some of the amount of the material to the second target; and (b4) at 240, removing the second coating from the distal tip. The method can include further similar cycles of picking and transferring more materials from a source to a target, reusing the picking pin in each cycle, wherein in each cycle the distal tip of the picking pin is coated with a fresh coating and then the coating is stripped away to prepare for the next cycle.

The material to be picked from the source can comprise a biological entity as described herein. For example, the source may comprise a culture media including one or more cells. In this case, the material picked and transferred in the process can be one or more cells, such as one or more eukaryotic cells or bacterial cells. What is retained in the pinking pin during the transfer process can be an aliquot of liquid with the cells entrained therein.

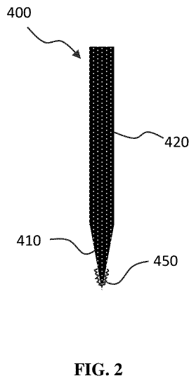

A schematic depiction of a portion of a picking pin 400 is shown in FIG. 2, which includes a distal tip 410 (partially coated with a coating of organic molecules 450 as will be described further below). The substrate of the pin (without the coating described herein) can be made from of a metal or metal alloy such as tungsten or a tungsten alloy. The pin can include a stem portion 420 having a substantially cylindrical shape, and the distal tip 410 can have a substantially cone shape, where the tip of the cone is small enough to be accommodated by the source container or compartment thereof. The stem portion 420 and distal tip 410 can also take other configurations as suitable or desired. The coating of organic molecules can be coated on part of the distal tip 410, or can be coated on the distal tip as well as part of the stem portion 420.

The coating of organic molecules can comprise natural macromolecules such as proteins, peptides, polysaccharides, lipids, etc., or synthetic oligomers or polymers. In some embodiments, the coating can include a hydrogel-forming natural polymer such as collagen and gelatin and polysaccharides such as starch, alginate, and agarose. The polymers can include those polymers having hetero-atoms on the backbone or side branch or chains. In some embodiments, the organic molecules can include a film forming material, e.g., reactive oligomers such as phenol-formaldehyde, epoxy, and polyester resins, as well as non-reactive polymers such as chlorinated polyvinyl chloride resins, polyacrylates, and cellulose nitrates. In some embodiments, the coating can be poly(vinyl alcohol) (or PVA), which is a synthetic hydrogel-forming polymer due to its high affinity to water.

The coating process can be carried out by dipping the tip of the picking pin in a solution containing the organic molecules for a duration of time. The duration of time depends on the nature of the organic molecules, concentration of the organic molecules, affinity or reactivity of the organic molecules toward the metallic surface, thickness of the coating desired, etc. In some embodiments, it is desirable to complete the coating process quickly, e.g., within a few seconds. To meet this objective, the variables mentioned above can be adjusted.

In some embodiments, the coating process can include dipping into the pin tip in a polymer solution and in a solution containing a crosslinker suitable to crosslink the polymer. In one example, the pin tip is dipped in a PVA aqueous solution, and then the tip is dipped in a sodium borate solution. Sodium borate here is used to crosslink PVA, forming a more robust hydrogel of PVA (shown below is the crosslinking reaction).

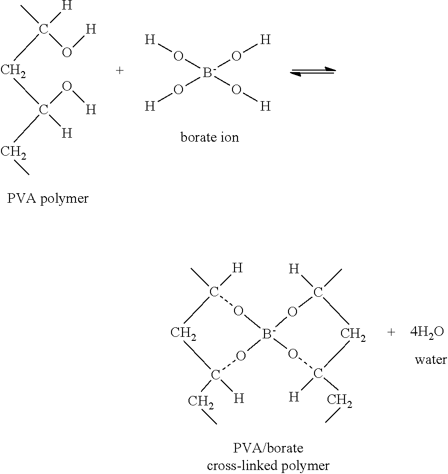

Alternatively, the pin can be first dipped into the crosslinking solution, and then dipped into the polymer to be crosslinked. Different concentrations of PVA and borate, PH, and other parameters can be adjusted to change the viscosity of the gel and the amount of material to be retained on the pin tip. The coated hydrogel layer can help retain much greater volume of the liquid than the bare metal surface.

In some embodiments, a hydrophobic coating can be applied on the picking pin. For example, the pin can be dipped into a PDMS water-based solution. The pin is then rinsed with water and allowed to dry for a few seconds. This coating is shown to improve the transfer rate of a soil sample to 60-85% while incurring no false positives.

The source (or source location) can be same or different in the multiple picking cycles. In some embodiments, the source can be contained in a first microwell of a microfabricated chip, and the second source (for the second cycle of picking) is contained in a second microwell of the microfabricated chip.

In some embodiments, the target can be a compartment of culture platform such as a petri dish, a 96-well plates or 384-well plates, or well(s) of a microfabricated chip or other culture platform. The target may include a culture media comprising liquid. The release of the retained material can be accomplished by leaving the material-loaded pin tip in the target for a given period or time, optionally with swirling or dithering the pin to expedite the release. In each picking-release operation cycle, the target can be same or different depending on the purpose and/or design of the material transfer.

After the picked material is released in the target, the coating is removed (together with any remnants of the picked material) so that the pin is ready for the next pick. The removal can be accomplished by moving the pin tip to a high-temperature (sterilization or heating) zone, e.g., more than 250° C. or higher temperature, for a duration of time, to effectively “burn off” the coating. In some embodiments, chemicals can be used to dissolve or otherwise break up the coating so as to remove the coating. For example, as the reaction of PVA with borate is well known, and acid can reverse the crosslinking. In this case, an acid bath can be used to clean off the pin with an eye towards acid that is weak/safe enough not to etch the pin or become a safety hazard. Acetic acid has been shown to work well. Dipping the pin into weak (100 mM) acetic acid for a few seconds to completely remove the crosslinked PVA coating from the pin tip. High temperature and chemical bath/rinse can be used in combination to remove the coating.

As used herein, a microfabricated device or chip may define a high density array of microwells (or experimental units). For example, a microfabricated chip comprising a “high density” of microwells may include about 150 microwells per cm² to about 160,000 microwells or more per cm² (for example, at least 150 microwells per cm², at least 250 microwells per cm², at least 400 microwells per cm², at least 500 microwells per cm², at least 750 microwells per cm², at least 1,000 microwells per cm², at least 2,500 microwells per cm², at least 5,000 microwells per cm², at least 7,500 microwells per cm², at least 10,000 microwells per cm², at least 50,000 microwells per cm², at least 100,000 microwells per cm², or at least 160,000 microwells per cm²). A substrate of a microfabricated chip may include about or more than 10,000,000 microwells or locations. For example, an array of microwells may include at least 96 locations, at least 1,000 locations, at least 5,000 locations, at least 10,000 locations, at least 50,000 locations, at least 100,000 locations, at least 500,000 locations, at least 1,000,000 locations, at least 5,000,000 locations, or at least 10,000,000 locations. The arrays of microwells may form grid patterns, and be grouped into separate areas or sections. The dimensions of a microwell may range from nanoscopic (e.g., a diameter from about 1 to about 100 nanometers) to microscopic. For example, each microwell may have a diameter of about 1 μm to about 800 μm, a diameter of about 25 μm to about 500 μm, or a diameter of about 30 μm to about 100 μm. A microwell may have a diameter of about or less than 1 μm, about or less than 5 μm, about or less than 10 μm, about or less than 25 μm, about or less than 50 μm, about or less than 100 μm, about or less than 200 μm, about or less than 300 μm, about or less than 400 μm, about or less than 500 μm, about or less than 600 μm, about or less than 700 μm, or about or less than 800 μm. In exemplary embodiments, the diameter of the microwells can be about 100 μm or smaller, or 50 μm or smaller. A microwell may have a depth of about 25 μm to about 100 μm, e.g., about 1 μm, about 5 μm, about 10 μm, about 25 μm, about 50 μm, about 100 μm. It can also have greater depth, e.g., about 200 μm, about 300 μm, about 400 μm, about 500 μm. The microfabricated chip can have two major surfaces: a top surface and a bottom surface, where the microwells have openings at the top surface. Each microwell of the microwells may have an opening or cross section having any shape, e.g., round, hexagonal, or square. Each microwell may include sidewalls. For microwells that are not round in their openings or cross sections, the diameter of the microwells described herein refer to the effective diameter of a circular shape having an equivalent area. For example, for a square shaped microwell having side lengths of 10×10 microns, a circle having an equivalent area (100 square microns) has a diameter of 11.3 microns. Each microwell may include a sidewall or sidewalls. The sidewalls may have a cross-sectional profile that is straight, oblique, and/or curved. Each microwell includes a bottom which can be flat, round, or of other shapes. The microfabricated chip (with the microwells thereon) may be manufactured from a polymer, e.g., a cyclic olefin polymer, via precision injection molding or some other process such as embossing. The chip may have a substantially planar major surface.

The high density microwells on the microfabricated chip can be used for receiving a sample comprising at least one biological entity (e.g., at least one cell). The term “biological entity” may include, but is not limited to, an organism, a cell, a cell component, a cell product, and a virus, and the term “species” may be used to describe a unit of classification, including, but not limited to, an operational taxonomic unit (OTU), a genotype, a phylotype, a phenotype, an ecotype, a history, a behavior or interaction, a product, a variant, and an evolutionarily significant unit. The high density microwells on the microfabricated chip can be used to conduct various experiments, such as growth or cultivation or screening of various species of bacteria and other microorganisms (or microbes) such as aerobic, anaerobic, and/or facultative aerobic microorganisms. The microwells may be used to conduct experiments with eukaryotic cells such as mammalian cells. Also, the microwells can be used to conduct various genomic or proteomic experiments, and may contain cell products or components, or other chemical or biological substances or entities, such as a cell surface (e.g., a cell membrane or wall), a metabolite, a vitamin, a hormone, a neurotransmitter, an antibody, an amino acid, an enzyme, a protein, a saccharide, ATP, a lipid, a nucleoside, a nucleotide, a nucleic acid (e.g., DNA or RNA), a chemical, e.g., a dye, enzyme substrate, etc.

In various embodiments, a cell may be Archaea, Bacteria, or Eukaryota (e.g., fungi). For example, a cell may be a microorganism, such as an aerobic, anaerobic, or facultative aerobic microorganisms. A virus may be a bacteriophage. Other cell components/products may include, but are not limited to, proteins, amino acids, enzymes, saccharides, adenosine triphosphate (ATP), lipids, nucleic acids (e.g., DNA and RNA), nucleosides, nucleotides, cell membranes/walls, flagella, fimbriae, organelles, metabolites, vitamins, hormones, neurotransmitters, and antibodies.

For the cultivation of cells, a nutrient is often provided. A nutrient may be defined (e.g., a chemically defined or synthetic medium) or undefined (e.g., a basal or complex medium). A nutrient may include or be a component of a laboratory-formulated and/or a commercially manufactured medium (e.g., a mix of two or more chemicals). A nutrient may include or be a component of a liquid nutrient medium (i.e., a nutrient broth), such as a marine broth, a lysogeny broth (e.g., Luria broth), etc. A nutrient may include or be a component of a liquid medium mixed with agar to form a solid medium and/or a commercially available manufactured agar plate, such as blood agar.

A nutrient may include or be a component of selective media. For example, selective media may be used for the growth of only certain biological entities or only biological entities with certain properties (e.g., antibiotic resistance or synthesis of a certain metabolite). A nutrient may include or be a component of differential media to distinguish one type of biological entity from another type of biological entity or other types of biological entities by using biochemical characteristics in the presence of specific indicator (e.g., neutral red, phenol red, eosin y, or methylene blue).

A nutrient may include or be a component of an extract of or media derived from a natural environment. For example, a nutrient may be derived from an environment natural to a particular type of biological entity, a different environment, or a plurality of environments. The environment may include, but is not limited to, one or more of a biological tissue (e.g., connective, muscle, nervous, epithelial, plant epidermis, vascular, ground, etc.), a biological fluid or other biological product (e.g., amniotic fluid, bile, blood, cerebrospinal fluid, cerumen, exudate, fecal matter, gastric fluid, interstitial fluid, intracellular fluid, lymphatic fluid, milk, mucus, rumen content, saliva, sebum, semen, sweat, urine, vaginal secretion, vomit, etc.), a microbial suspension, air (including, e.g., different gas contents), supercritical carbon dioxide, soil (including, e.g., minerals, organic matter, gases, liquids, organisms, etc.), sediment (e.g., agricultural, marine, etc.), living organic matter (e.g., plants, insects, other small organisms and microorganisms), dead organic matter, forage (e.g., grasses, legumes, silage, crop residue, etc.), a mineral, oil or oil products (e.g., animal, vegetable, petrochemical), water (e.g., naturally-sourced freshwater, drinking water, seawater, etc.), and/or sewage (e.g., sanitary, commercial, industrial, and/or agricultural wastewater and surface runoff).

After a sample is loaded on a microfabricated device, a cover film or membrane may be applied to at least a portion of a microfabricated device. For example, after a sample is loaded on a microfabricated device, at least one membrane may be applied to at least one microwell of a high density array of microwells. A plurality of membranes may be applied to a plurality of portions of a microfabricated device. For example, separate membranes may be applied to separate subsections of a high density array of microwells. A membrane may be connected, attached, partially attached, affixed, sealed, and/or partially sealed to a microfabricated device to retain at least one biological entity in the at least one microwell of the high density array of microwells. For example, a membrane may be reversibly affixed to a microfabricated device using lamination. A membrane may be punctured, peeled back, detached, partially detached, removed, and/or partially removed to access at least one biological entity in the at least one microwell of the high density array of microwells.

In some embodiments, the population of cells in at least one experimental unit, well, or microwell in the microfabricated chip may be picked by puncturing the cover film with a picking pin with a coating as described herein.

A membrane or cover film may be impermeable, semi-permeable, selectively permeable, differentially permeable, and/or partially permeable to allow diffusion of at least one nutrient into the at least one microwell of a high density array of microwells. For example, a membrane may include a natural material and/or a synthetic material. A membrane may include a hydrogel layer and/or filter paper. In some embodiments, a membrane is selected with a pore size small enough to retain at least some or all of the cells in a microwell. For mammalian cells, the pore size may be a few microns and still retain the cells. However, in some embodiments, the pore size may be less than or equal to about 0.2 μm, such as 0.1 μm. An impermeable membrane has a pore size approaching zero. It is understood that the membrane may have a complex structure that may or may not have defined pore sizes.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed.

1. A method of transferring material using a picking pin having a distal tip, comprising:
(a1) forming a first coating of organic molecules on at least a portion of the distal tip of the picking pin;
(a2) dipping at least a portion of the coated distal tip in a first source such that an amount of material is picked up from the first source and attached to the first coating;
(a3) dipping the coated distal tip attached with the amount of material into a first target so as to release some of the amount of the material to the first target; and
(a4) removing the first coating from the distal tip.

2. The method of claim 1, further comprising:
(b1) forming a second coating of organic molecules on at least a portion of the distal tip of the picking pin, the second coating being the same as or different from the first coating;
(b2) dipping at least a portion of the distal tip in a second source such that an amount of material is picked up from the second source and attached to the second coating;
(b3) dipping the distal tip attached with the amount of material into a second target so as to release some of the amount of the material to the second target, the second target being the same as or different from the first target; and
(b4) removing the second coating from the distal tip.
